(19) United States
(12) Patent Application Publication
(10) Pub. No.: US 2003/0069220 A1
(43) Pub. Date: Apr. 10, 2003

(54) METHOD FOR MASS MEDICATION IN BIOLOGICAL ATTACKS

(76) Inventor: Michael Strobel, Northfield, MN (US)

Correspondence Address: John W. Ryan, WILMER CUTLER & PICKERING, 2445 M. Street, N.W., Washington, DC, 20037-1420, US

(21) Appl. No.: 09/963,741

(22) Filed: Sep. 27, 2001

(51) Int. Cl. A61K 31/545; A61K 31/19

(52) U.S. Cl. 514/200; 514/574

(57) ABSTRACT

A method of treating a plurality of individuals exposed to a biological agent is disclosed. The method includes distributing a sufficient amount of an aqueous solution. The aqueous solution is a palatable concentration of a cephalosporin or its equivalent mixed with a specified hydroxypolycarboxylic acid. The aqueous solution having a weight ratio of the hydroxypolycarboxylic acid to cephalosporin or its equivalent of at least about 1.8.

### FIELD OF THE INVENTION

This invention relates generally to a method for mass medication in biological attacks, and more particularly although not exclusively to mass medication of the population exposed to anthrax, bubonic plague, and/or cholera.

### BACKGROUND OF THE INVENTION

Terrorism is a clear and present danger to Americans today. Generally, the terrorist attacks have utilized explosives, however, biological attacks remain a distinct possibility. Multiple studies by both government and privately owned institutions have described the widespread devastation to society that a successful biological attack may cause.

Anthrax (Bacillus anthracis), bubonic plague (Yersinia pestis), and cholera (Vibrio cholerae) in particular are known biological agents that have been demonstrated to be effective weapons of mass destruction.

Depending on the specific biological agent, various treatments with erythromycin, doxycyclene, penicillin, cephalosporins, tetracycline and/or streptomycin have proven to be effective in reducing morbidity and mortality of individuals infected with biological agents. However, prompt treatment is generally critical to increasing survival rates.

Bacillus anthracis, the causative organism for anthrax, is a large, gram-positive, facultatively anaerobic, encapsulated rod. The spores resist destruction and remain viable for long periods of time and in adverse conditions. Inhaling spores of this organism may result in pulmonary anthrax which is often fatal. Pulmonary anthrax is characterized by the rapid multiplication of the organism in the mediastinal lymph nodes. Severe hemorrhagic necrotizing lymphadenitis develops and spreads throughout the lungs. Initial symptoms are insidious and resemble influenza. Fever increases within a few days followed by shock and coma. Without early diagnosis and treatment, a fatal outcome may ensue. Treatment typically consists of penicillin and/or cephalosporins.

Yersinia pestis, the causative organism of the plague (e.g., Bubonic plague, Black death, etc.), is a short bacillus. The plague may be spread by inhalation of infectious droplets or dust. Symptoms include enlarged lymph nodes, restlessness, deliria, confusion and lack of coordination. A cough may develop; sputum is mucoid at first, however, bloody sputum rapidly develops. Most untreated patients die within 48 hrs after onset of symptoms. Treatment should be immediate upon suspicion of the plague. Recommended treatments include tetracycline, chloramphenicol and/or cephalosporins.

Vibrio cholerae, the causative agent of cholera, is a short, curved, motile, aerobic rod. Cholera is spread by ingestion of infected water. The manifestations of cholera result from the loss of isotonic, watery stools rich in sodium, chloride, etc. If untreated, circulatory collapse and stupor may follow. The fatality rate may exceed 50% if untreated, however, it may be reduced below 1% if prompt treatment is administered. Early treatment with tetracycline and/or cephalosporins is recommended.

Cephalosporins thus are capable of reducing the morbidity and mortality through early treatment of an exposed population. Cephalosporins comprise the class of amphoteric antibiotics of the group of broad-spectrum, relatively penicillinase-resistant antibiotics derived from the fungus Cephalosporium, which share the nucleus 7-aminocephalosporanic acid. They are related to the penicillins in both structure and mode of action. Their antibacterial activity results from inhibition of the cross-linking of peptidoglycan units in the cell wall. The cephalosporins available for medicinal use are semisynthetic derivatives of the natural antibiotic cephalosporin C. The cephamycins cefotetan and cefoxitin and the β-lactam moxalactam are included with the cephalosporins because of their dose relationship to them. Cephalosporin C is isolated from Cephalosporium acremonium, which is the parent compound of a number of semisynthetic antibiotics, including cefazolin sodium, cephalexin (αaminobenzyl-3-desacetoxycephalosporin), cephaloridine, cephaloglycin, cephalothin, cephapirin and cephradine (α-amino-2,5-dihydrobenzyl-3-desacetoxycephalosporin), used in the treatment of a wide variety of infections due to sensitive gram-positive and gram-negative bacteria. The first generation cephalosporins have a broad range of activity against gram-positive organisms and a narrow range of activity against gram-negative organisms and include cephalothin, cefazolin, cephaloridione, cephapirin, cephadrine, cephalexin, and cefadroxil. The second generation cephalosporins are more active against gram-negative organisms and less active against gram-positive organisms than the first-generation agents. They include cefamanadole, cefoxitin, cefaclor, and cefurotrime. The third-generation cephalosporins are a group of β-lactamase-resistant cephalosporins that are more active against gram-negative organisms and less active against gram-positive organisms than second-generation agents. They include cefoperazone, cefotaxime, ceftriaxone, ceftazidime, ceftizoxime, and moxalactam. Collectively, all fall under the label of cephalosporin, and as that term is used herein and in the claims, the term covers any one or more of these various generation agents.

Cephalexin is a widely employed antibiotic. It is effective against a wide range of gram-negative and gram-positive bacteria. It is used in the treatment of infections of the urinary and respiratory tracts and of skin and soft tissues due to sensitive pathogens. In treating humans, it is typically administered orally, e.g., in a tablet form.

The treatment of humans with a cephalosporin or its equivalent such as cephalexin is fraught with many difficulties. For example, injecting cephalexin is time consuming and costly, especially when large numbers of people are involved. Distributing tablets or setting up injection sites for administration of a cephalexin compound is minimally a taxing chore. Mixing solid particles of cephalexin in the food supply assures uneven dosage of the medication and, because cephalexin is unpalatable, creates a bad tasting mixture that the general population is less likely to ingest. Because cephalexin is water insoluble, it cannot be dissolved in the water supply. However, when cephalexin is made water soluble by derivatization according to the prior art, its aqueous solutions typically suffer from poor shelf-life stability, sometimes having a shelf-life of only a few hours, and unpalatability, causing the general population to reduce their ingestion of water containing the unpalatable form of cephalexin.

Cephalexin readily dissolves in the acidic environment of the normal stomach, e.g., pH of less than 3.0. It is available in the form of suspensions and tablets (such as cephalexin monohydrate), an unstable water-soluble form of sodium cephalexin that is stable for less than about 6 hours, and a water soluble form of cephalexin hydrochloride (such as cephalexin hydrochloride monohydrate, see U.S. Pat. Nos. 4,600,773 and 4,775,751) that is sold in coated tablet form (Keftab™ from Eli Lilly and Company). Aqueous solutions of sodium cephalexin and cephalexin hydrochloride are unpalatable.

Cephalexin hydrochloride and cephalexin monohydrate are readily dissolved in forms which are 50% positively charged and 50% neutrally charged. The neutrally charged form has limited solubility compared to the ionized drug forms. At a pH 4.5, cephalexin monohydrate exists as 100% neutrally charged particles, with limited solubility. However, cephalexin hydrochloride would be expected to be more soluble because of the hydrochloride moiety.

Aszodi, et al. U.S. Pat. No.5,728,828, patented Mar. 17, 1998 describe a variety of compositions of cephalosporins having a very good antibiotic activity on gram (+) bacteria such as staphylococci, streptococci and notably on penicillin-resistant staphylococci. Their effectiveness on gram (−) bacteria, particularly on coliform bacteria, klebsiella, salmonella, proteus and pseudomonas, is particularly remarkable. Further, these properties make the compositions suitable for use as medicaments in the treatment of germ-sensitive diseases and particularly in that of staphylococci, such as staphylococci septicemias, malignant staphylococcia of the face or skin, pyodermititis, septic or suppurative sores, anthrax.

Dursch, U.S. Pat. No.3,940,483, patented Feb. 24, 1976, describes dry solid antibiotic compositions of a solid acidic, basic or amphoteric antibiotic, inclusive of cephalosporin or its equivalent, such as cephalexin, and a suitable solid basic or acidic additive, for reconstitution as injectables upon addition of water. According to this patent, antibiotics of limited water solubility are formulated for parenteral application either as aqueous suspensions, or by preparing water soluble derivatives (e.g., salts, esters or complexes) of the parent compound, which upon parenteral administration are either in equilibrium with the parent compound, or which are transformed back into the parent compound in the patient's system. The patentee states that a solids in suspension “severely limits the mode of parenteral administration.” The patentee also states that the pharmaceutically acceptable solid derivatives are frequently prepared with significant yield losses. The derivatives are alleged to resist isolation in suitable form altogether. The patentee overcomes the prior art deficiencies by premixing antibiotics of limited water solubility which are either acidic, basic, or amphoteric in nature, with a suitable solid additive to form a dry mixture. On addition of water to the dry mixture, physiologically acceptable solutions of water soluble salts of the antibiotic are formed in situ and can be administered without delay.

The patentee illustrates antibiotics such as penicillins, e.g., ampicillin, cephalosporins, e.g., cephalexin, and the like, as amphoteric antibiotics that can be dry blended. According to this patent, “suitable additives for amphoteric antibiotics may be of either acidic or basic character.” illustrative of suitable acidic additives are alkali metal hydrogen sulfates, and organic acids like citric acid, tartaric acid or maleic acid. There is no explicit indication of the amount of acid compound that is to be used in the dry mixture. In this regard, the patentee states: “The selected solid additive is usually employed in an amount just sufficient to assure complete dissolution of the antibiotic upon addition of a small volume of water. This amount may well be less than the stoichiometric quantity required for complete conversion to a salt. Herein lies another advantage of the present invention over the use of pre-formed salts; frequently, less extreme conditions of acidity or basicity are required for complete dissolution and superior stability of such solutions can be expected. For example, 95 mole- % of sodium carbonate is sufficient to dissolve ampicillin at pH 8.3, whereas an aqueous solution of pre-formed sodium ampicillin shows about pH 9.5.” The examples of the patent show dry mixtures that on dissolution in water result in pH's as low as 2 and as high as 9.7. The patent is unconcerned with ingestion of the antibiotic or for forming a palatable dry mixture that can be ingested by the population.

French patent publication 2 308 368 describes a lysine salt of cephalexin that can be administered orally as well as by intramuscular or intravenous injection. Such a lysine salt dissolved in water has been found to be unpalatable.

There is a need for stable forms of cephalosporins and their equivalents, especially cephalexin, that can be orally administered (i.e., ingested) via the drinking water supply to address the present threat of a biological terrorist attack using Bacillus anthracis, Yersinia pestis, and/or Vibrio cholerae. Palatable forms of cephalosporins and their equivalents, especially the widely used cephalexin, allows large scale dosage-controlled treatment of populations with the antibiotic. An unpalatable soluble form of cephalexin affects the taste of the drinking water to a degree that jeopardizes the amount of cephalexin ingested by the population thus making it impossible to accurately control dosage to the population.

### SUMMARY OF THE INVENTION

The invention pertains to a method for medication of a population exposed to a biological agent. The method includes distributing a sufficient amount of an antibiotic powder (including cephalosporin or its equivalent and a hydroxypolycarboxylic acid) or antibiotic aqueous solution (including cephalosporin or its equivalent and a hydroxypolycarboxylic acid) to substantially reduce morbidity and mortality in the population by the biological agent. The antibiotic powder (or antibiotic aqueous solution) being readily soluble in water. The antibiotic aqueous solution is stable for at least 7 days whereas the antibiotic powder is stable indefinitely.

A method of treating a population of individuals exposed to a biological agent using at least one readily water-soluble ingestible form of cephalosporins or their equivalents (e.g., cephalexin, cephalothin, cephaloridine, and certain newer generation broad spectrum antibiotics) formed by their reaction with a hydroxylatedpolycarboxylic acid. This water-soluble form possesses palatability and unique storage stability. The water-soluble form of the invention allows distribution via municipal water supplies to humans, as well as animals, without loss of antibiotic benefits. This water-soluble form is derived from an anhydrous solid, particulate mixture of the cephalosporin or its equivalent and the hydroxylatedpolycarboxylic acid.

The invention further pertains to a method for medication of a population exposed to a biological agent. The method includes distributing a sufficient amount of an antibiotic mixture to substantially reduce morbidity and mortality in the population by the biological agent. The antibiotic mixture is readily soluble in water and is palatable. The antibiotic mixture includes at least one member of a cephalosporin class of antibiotics.

In comparison to known prior art, certain embodiments of the invention are capable of achieving certain aspects, including some or all of the following: (1) quickly distributing vast amounts of antibiotics to a population; (2) treating individuals that are unaware they have been exposed; (3) increasing the number of medicine distribution cites; and (4) treating individuals in their homes thus reducing further spread of disease. Those skilled in the art will appreciate these and other aspects of various embodiments of the invention upon reading the following detailed description of a preferred embodiment.

### DETAILED DESCRIPTION OF THE INVENTION

This invention relates to the treatment of one or more individuals exposed to a biological agent with an aqueous solution comprising a palatable concentration of a cephalosporin or its equivalent and a hydroxypolycarboxylic acid of the formula:

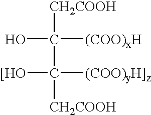

where x and y are 0 or 1 and z is 0 to 3 in the weight ratio of the hydroxypolycarboxylic acid to cephalosporin or its equivalent of at least about 1.8. Optionally, a sweetening agent (e.g., sugar and the like) and/or a flavoring agent may be added to the antibiotic aqueous solution. Furthermore, an anhydrous mixture of the antibiotic aqueous solution, with or without the optional sweetening agent and/or a flavoring agent, may also be utilized in the practice of various embodiments of the invention. A more detailed description of the use of the hydroxypolycarboxylic acid mentioned above in conjunction with various cephalosporins may be found in the U.S. Pat. No. 5,721,229, the disclosure of which is hereby incorporated by reference in its entirety.

Specifically, the invention relates to the treatment of a population having been exposed to biological agents by terrorist. However, as is known to those skilled in the art, various embodiments of the invention have implications in treating the population exposed to naturally occurring biological organisms and thus are within the scope of the invention. In a preferred example, the antibiotic aqueous solution may be distributed to the population via a municipal water supply in response to a biological attack by terrorist. The antibiotic powder being mixed into the municipal water supply and carried via existing water supply lines to the population in an effected area. In various other embodiments, the antibiotic powder and/or the antibiotic aqueous solution may be distributed via a plurality of vehicles to a multitude of distribution centers. Regardless of the manner of distribution, it may be preferable to increase the number of distribution cites so as to provide rapid distribution and usage so as to prevent the spreading of disease.

The cephalosporins and their equivalents used in the practice of this invention are those that are antibacterials to the biological agent(s) infecting the population. The term “anhydrous,” when used to characterize the solid mixture of the cephalosporins and their equivalents, does not exclude bound water in the form of hydrated water bound to the cephalosporins and their equivalents. For the purposes of this invention, even when a cephalosporin or its equivalent hydrate or its equivalent such as cephalexin monohydrate is provided in the mixture, the mixture is still regarded to be anhydrous. The reason for this characterization is based on the fact that the hydrate form of the cephalosporins and their equivalents remains solid and dry and can be formed into the mixture without forming a solution.

An important facet of the invention is the weight ratio of hydroxypolycarboxylic acid to the cephalosporin or its equivalent in the mixture. It has been determined that the weight ratio of anhydrous solid hydroxypolycarboxylic acid to solid cephalosporin and its equivalent (typically in the hydrate form) should be at least about 1.8 to 1, preferably at least 2.5 to 1, most desirably about 3 to 1, in the mixture in order to readily form, in combination with tap, deionized or distilled water, a palatable solution that can be administered to humans without rejection. The highest weight ratio of hydroxypolycarboxylic acid to cephalosporin or its equivalent may be about 10 to 1, though it is preferred to keep the weight ratio below about 5 to 1 of hydroxypolycarboxylic acid to cephalosporin or its equivalent. At a lower weight ratio, the resultant cephalosporin or its equivalent and hydroxypolycarboxylic acid mixture does not form a stable solution, and in many cases, will not form a solution. In all cases tested, when attempting to form a solution from a hydroxypolycarboxylic acid/cephalosporin or its equivalent mixture having a weight ratio less than 1.8, to the extent a solution is formed, the solution is clearly unpalatable.

In addition, in order to form a palatable aqueous solution of the hydroxyacylated cephalosporin or its equivalent, the hydroxyacylated cephalosporin or its equivalent solution has a pH of about 3.0 to as high as about 4.0, preferably from about 3.3 to about 3.8.

To enhance the palatability of the solution, one may add flavorings and/or artificial sweeteners such as cyclohexylsulfamic acid, saccharin (o-benzosulfimide), and Aspartame (i.e., L-Aspartyl-L-phenylalanine methyl ester) sold as Nutrasweet™ artificial sweetener, and the like, in small amounts that are sufficient to enhance the palatability of the hydroxyacylated cephalosporin or its equivalent solution. Additionally, sugar can be used to sweeten the solution. In actual practice, the sweetener and the flavoring are added in amounts that overcome the natural bad taste of the cephalosporins and their equivalents that is not fully mitigated by the presence of the hydroxypolycarboxylic acid. Furthermore, the presence of flavorings and/or sweeteners may serve as an indicator the water has been treated. Thus, municipal workers may purge existing, untreated water from supply lines until the flavorings are detected. Moreover, individuals may purge existing, untreated water from their homes until the flavorings are detected. In this manner, the population may be reassured that they are receiving treatment.

Cephalexin, as an example, is normally 90% absorbed by the body when ingested. The dosage of the antibiotic powder used in forming an antibiotic aqueous solution for ingestion by the population is quite broad, and should be sufficient to provide the required amount of cephalosporins and their equivalents prescribed by the medical community. As a rule, the amount of the antibiotic powder that is dissolved into water for distribution to the population should provide about 2 to about 15, preferably from about 5 to about 10, milligrams of the cephalosporin or its equivalent for each pound of weight of the persons and/or animals being treated.

Various embodiments of the invention allow for the treatment of the population with at least one preparation of the antibiotic powder in the weight ratio of 1.8 to 5.0 of the hydroxypolycarboxylic acid to 1 of the cephalosporin or its equivalent. The mixture may contain from about 0.1 to about 5 weight percent of a flavoring and from about 0.1 to about 10 weight percent of an artificial sweetener. In special situations, greater and smaller amounts of the hydroxypolycarboxylic acid, flavoring and artificial sweetener can be used to advantage to achieve the objectives of this invention. The antibiotic powder is stable and can be kept for an infinite period of time without deterioration. Contrary to the normal attributes of prior art acidic solutions of cephalosporins and their equivalents, the antibiotic aqueous solutions of the invention have remarkable storage stability as compared to non-acidified cephalosporins and their equivalents such a cephalexin monohydrate, and the like. These antibiotic aqueous solutions can be kept for as long as 14 days, at refrigeration conditions of about 7° C., without deterioration or separation (e.g., precipitation of cephalosporin or its equivalent). In a preferred form, 1 gram of Cephalexin or equivalent may be mixed with 3 grams of citric acid or an equivalent amount of an amino acid. Flavorings and sweeteners may be added at a level sufficient for palatability.

The mixing of the antibiotic powder, with or without flavoring and sweetener, is not difficult to achieve. Any method and equipment that is effective in making a powdered mixture can be used in the practice of this invention to produce the antibiotic powder. For example, small packets of the antibiotic powder can be put into a plastic bag, typically one made of polyethylene, or polyvinyl chloride-vinylidene chloride copolymer or Mylar™ (a film form of polyethyleneterephthalate), and the like. The antibiotic powders can be made in such bags by separately feeding solid particulate cephalosporin or its equivalent and solid particulate hydroxylatedpolycarboxylic acid. Then the ingredients in the bag can be worked by hand to mix the particulates into an essentially homogeneously distributed mixture. The particulates can be mixed in large and small scale mixing equipment. They can be mixed in a household cake mixer, a large scale Banbury Mixer, large and small paddle mixers, and the like. In this manner, households not served by municipal water supplies may receive distributions of the mixture. Additionally or alternatively, the mixture may be distributed to restaurants, soup kitchens and the like in large containers such as sacks for distribution to the population. However, in a preferred form, the antibiotic powder may be mixed into the municipal supply of water. For example, if a sufficient amount of the antibiotic powder is mixed into the municipal water supply to produce a concentration of 4 grams of cephalexin or the equivalent per gallon of water, this would deliver 500 mg of antibiotic per 16 ounces of drinking water or 250 mg of antibiotic per 8 ounces of drinking water. In this manner, the consumption of one 16 ounce glass of water every 8 hours may be sufficient to cure an adult infected with anthrax. Similarly, the consumption of one 8 ounce glass of water every 8 hours may be sufficient to cure a child under 12 years of age infected with anthrax. Obviously, dissolving less antibiotic powder into the water supply would require the consumption of a correspondingly greater amount water.

Furthermore, relatively large amounts of the antibiotic powder may be stored in one or more locations in the vicinity of municipalities servicing populations. In this regard, relatively large distribution centers may be set up in various locations in the vicinity of population centers of America (e.g., cities and towns). Thus, the citizens of America may be protected in our ongoing war against terrorism.

The antibiotic powder is preferably in the form of an intimate mixture of the cephalosporin or its equivalent and the hydroxypolycarboxylic acid particles. Preferably, the cephalosporin or its equivalent and the hydroxypolycarboxylic acid are each in the form of ready dissolvable powders. Such powders may be termed as fine grain powders comparable to granular or powdery sugar. The degree of blending of these powders is not critical so long as the right proportions of each component is dissolved in the body of water that is used in forming the solution comprising the cephalosporin or its equivalent and the hydroxypolycarboxylic acid. The key to blending and aliquoting the blend is to assure that the proper concentration of each component is present in the aliquot when the aliquot is dissolved in the municipal water supply or a common body of water.

The antibiotic powder may be dissolved in a small concentration of water, for example, an amount of water sufficient to dissolve all of the components of the dry mixture. This solution can be a concentrate of the cephalosporin or its equivalent and is used as a vehicle for supplying the ingredients of the antibiotic powder of the invention in the water supply.

### EXAMPLE 1

The following antibiotic powders were formed without sweeteners and flavorings by addition to clear plastic bags and by working of the bags. The mixtures were added to water in the amounts noted and the conditions of dissolution are noted, as well as the resultant pH or the formed solution. Although Example 1 describes the use of citric acid, various other constituents may be utilized as a sexualizing agent (e.g., various amino acids).

| Choice of | Choice of | | |
|---|---|---|---|
| cephalosporin or | polyhydroxylated- | | |
| equivalent and | polycarboxylic | Amount | |
| amount | acid and amount | of water | Comments |
| Cefadroxil | Citric Acid | 25 cc | Formed a clear yellow |
| 1 gram | 3 grams | | solution within 2 |
| | | | minutes having a pH |
| | | | of about 3. |
| Cephradine | Citric Acid | 25 cc | Formed a clear yellow |
| 1 gram | 3.5 grams | | solution in 15 |
| | | | minutes having a pH |
| | | | of about 3. |
| Cephaloridine | Citric Acid | 25 cc | Formed a clear solution |
| 1 gram | 3 grams | | in 8 minutes |
| | | | that has a pH |
| | | | of about 3. |
| Cefaclor | Citric Acid | 2.5 cc | Formed a solution |
| 0.1 grams | 0.3 grams | | in 12 minutes that |
| | | | has a pH of 2.8. |
| Cephalexin | Citric Acid | 50 cc | Formed a clear yellow |
| 5 grams | 10 grams | | solution in 5 |
| | | | minutes that has a |
| | | | pH of 3.3. |

In addition to those acids mentions above the following list of acids should also serve as solubilizing agents:

Lactic Acid and Salts i.e. Calcium Lactate

Malic acid

Sodium sulfate

Tarturik

Phosphoric Acid and Phosphate Salts

Aconitic Acid

Alum & Salts

Aluminum Sulfate

HCI

Sulfuric Acids

Ammonium C1

Ammonium Sulfate

Acetates and Salts

Calcium Acid Pyrophosphate

All Amino Acids and Salts

Calcium Chloride

Erythorbic Acid

Glucano Delta Lactone

Sodium Acetate

Cyclodextins

### EXAMPLE 2

A flavored and sweetened formulation of the invention is made by thoroughly mixing 1 kg of cephalexin monohydrate, 227 gms of Nutrasweet™ Aspartame sweetener, 3 kg of citric acid and 30 gms of strawberry flavoring. This anhydrous mixture is rapidly dissolved in water to a concentration of 128 grams of cephalexin per gallon of demineralized water to form a solution having a pH of about 3.5. A comparable solution is formed by substituting an equal weight amount of tartaric acid for the citric acid.

What has been described and illustrated herein is a preferred embodiment of the invention along with some of its variations. The terms, descriptions used herein are set forth by way of illustration only and are not meant as limitations. Those skilled in the art will recognize that many variations are possible within the spirit and scope of the invention, which is intended to be defined by the following claims—and their equivalents—in which all terms are meant in their broadest reasonable sense unless otherwise indicated.

What is claimed is:

1. A method of treating a plurality of individuals exposed to a biological agent comprising:
distributing a sufficient amount of an aqueous solution, the aqueous solution comprising a palatable concentration of a cephalosporin or its equivalent and a hydroxypolycarboxylic acid of the formula: 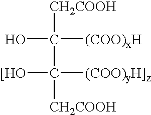
where x and y are 0 or 1 and z is 0 to 3 in the weight ratio of the hydroxypolycarboxylic acid to cephalosporin or its equivalent of at least about 1.8.

2. The method according to claim 1, wherein the aqueous solution is distributed via a municipal water supply, the municipal water supply providing water to the plurality of individuals.

3. The method according to claim 1, wherein the aqueous solution is distributed via a plurality of tanker trucks to the plurality of individuals.

4. The method according to claim 1, wherein an anhydrous mixture of the aqueous solution is distributed to the plurality of individuals.

5. The method according to claim 1, wherein the biological agent is Bacillus anthracis.

6. The method according to claim 1, wherein the biological agent is Yersinia pestis.

7. The method according to claim 1, wherein the biological agent is Vibrio cholerae.

8. A method for medication of a population exposed to a biological agent comprising the following steps:
mixing a cephalosporin or its equivalent and a hydroxypolycarboxylic acid of the formula: 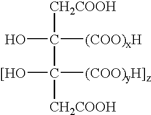
where x and y are 0 or 1 and z is 0 to 3 in the weight ratio of the hydroxypolycarboxylic acid to cephalosporin or its equivalent of at least about 1.8 to form a dry mixture;
optionally adding one or both of a sweetener and flavoring to the dry mixture;
storing the dry mixture at one or more predetermined designated locations; and
dissolving an effective amount of the dry mixture in a water supply.

9. The method according to claim 8, wherein the biological agent is Bacillus anthracis.

10. The method according to claim 8, wherein the biological agent is Yersinia pestis.

11. The method according to claim 8, wherein the biological agent is Vibrio cholerae.

12. The method according to claim 8, wherein water supply is a municipal water supply providing water to the population.

13. The method according to claim 8, wherein the water supply is distributed via a plurality of tanker trucks to the population.

14. The method according to claim 8, wherein the dry mixture is distributed to the population.

15. A method for medication of a population exposed to a biological agent comprising the following steps:
mixing a cephalosporin or its equivalent and a hydroxypolycarboxylic acid of the formula: 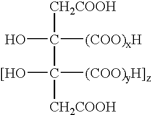
where x and y are 0 or 1 and z is 0 to 3 in the weight ratio of the hydroxypolycarboxylic acid to cephalosporin or its equivalent of at least about 1.8 to form an aqueous mixture;
optionally adding one or both of a sweetener and flavoring to the aqueous mixture;
storing the aqueous mixture at one or more predetermined designated locations; and
adding an effective amount of the aqueous mixture in a water supply.

16. The method according to claim 15, wherein the biological agent is Bacillus anthracis.

17. The method according to claim 15, wherein the biological agent is Yersinia pestis.

18. The method according to claim 15, wherein the biological agent is Vibrio cholerae.

19. The method according to claim 15, wherein water supply is a municipal water supply providing water to the population.

20. The method according to claim 15, wherein the water supply is distributed via a plurality of tanker trucks to the population.
